(19) United States
(12) Patent Application Publication
(10) Pub. No.: US 2011/0201878 A1
(43) Pub. Date: Aug. 18, 2011

(54) Self-inflating sexual aid

(76) Inventor: Cheng-Wu Chen, Lujhu Township (TW)

(21) Appl. No.: 12/656,797

(22) Filed: Feb. 17, 2010

(51) Int. Cl. A61F 5/00

(52) U.S. Cl. 600/38

(57) ABSTRACT

The present invention is to provide a self-inflating sexual aid, which includes a closed bag having an airtight sealed edge while a receiving space is formed therein, and a reactive powder and a sachet containing a reactive liquid inside the receiving space. When squeezed, the reactive liquid flows out of the sachet and contacts with the reactive powder to produce gaseous carbon dioxide and enable the closed bag to be automatically and instantly inflated to a completely taut state. An elastic cover has an inner surface that can be fitted closely over an outer surface of a rod-shaped portion of the closed bag, thus forcing the corresponding airtight sealed edge to lie smoothly against the outer surface of the rod-shaped portion. Consequently, a user can hold a handle portion of the closed bag and use the sexual aid by maneuvering the rod-shaped portion of the closed bag.

### FIELD OF THE INVENTION

The present invention relates to a sexual aid, more particularly to a self-inflating sexual aid, which is designed for women, can be automatically and instantly inflated to a completely taut state or be folded and downsized when not in use so as to facilitate carriage and storage, and can be directly discarded after use so as to spare its user the otherwise necessary cleanup work.

### BACKGROUND OF THE INVENTION

By the end of puberty, as the male and female reproductive system gradually matures, people begin to feel an intense sexual drive; hence, an unintentional rub at the genitals sometimes develops into an autosexual behavior, or masturbation, which for most people is also the first learned sexual behavior. While masturbation was seen as immoral or obscene in the traditional society, it is now viewed as helpful to mental as well as physical health, thanks to the advancement of medical science and an improvement in sexual knowledge.

In the past few decades, women's rights to education and work have reached the same level as men's. As a result, many women not only possess high academic degrees, but also work with such diligence and professionalism that they hold important posts in governments and private companies. Nevertheless, the traditional notion still prevails that women are supposed to do all the house chores such as doing the laundry, grocery shopping, and cooking, after a day's work. In consequence, modern women are divided between family and career and suffer from huge metal stress. Moreover, taking the married but separated couples across the Taiwan Strait for example, lack of sex life also results in a sense of loneliness or a melancholy feeling that impairs marital happiness and domestic harmony. Under such circumstances, autosexual behavior is an effective solution to relieving stress. During masturbation, the brain secretes a hormone that has an exhilarating effect, allowing women to temporarily get rid of such negative feelings as loneliness, emptiness, and depression and find mental and physical relief. Masturbation is also a natural way of sexual relief. As it is human nature to have sexual desires, autosexual behavior helps avoid some abnormal or socially unacceptable sexual behaviors and lower the risk of contracting sexually transmitted diseases.

In addition to having positive effects on women's mental health, autosexual behavior is also beneficial to female physical health. According to medical studies, when a woman's sexual arousal is not properly dealt with for a long time, venous congestion may take place and eventually lead to “chronic pelvic congestion syndrome”, which is often accompanied by chronic abdominal pain that tends to aggravate after prolonged standing. Such symptoms not only cause physical discomfort to women, but also may compromise their household life and work performance. Moderate masturbation relieves pelvic congestion and hence contributes to women's physical and mental well-being.

As mentioned earlier, autosexual behavior is physically and mentally advantageous to certain females and therefore should not be regarded from a moral point of view. In particular, masturbation serves to satisfy the sexual needs of celibate, widowed, or physically challenged women. For couples who are forced to separate for an extended period of time (e.g., those whose husbands work as seamen, geological explorers, etc.), masturbation also helps reduce extra-marital sex and maintain domestic stabilization. Therefore, as the society becomes more and more broad-minded, and due to the spread of proper sexual knowledge, an increasing number of manufacturers have begun developing various kinds of sexual aids that are designed specifically for women so as to enhance their satisfaction. Sexual aids are also doctors' major tools for treating those experiencing sexual dysfunction or an unhappy sex life. Besides, sexual aids can be used to substantially improve the quality of, and add interactive fun to, sex.

The conventional female sexual aids, though capable of producing positive effects on the physical and mental health of women and enabling emotional adjustment, are inconvenient to use. For instance, female sexual aids typically have a three-dimensional phallic shape to facilitate gripping and to suit the female genital structure, but such a design is also too bulky and rigid to be folded for storage. Therefore, not only is it difficult to put a conventional female sexual aid in a woman's cosmetic bag for easy carriage during a business or pleasure trip, but also the place where the female sexual aid is stored must be constantly checked to avoid being discovered, for masturbation is an extremely private behavior, and it is shameful, as it is traditionally taught, to make public a woman's desire to masturbate. In other words, to carry a conventional female sexual aid turns out to be a source of tremendous mental stress and distraction, which may in turn lower work or travel quality. In addition, since most female sexual aids nowadays are designed for repeated use, they must be washed and wiped immediately after each use and properly stored in a dry and clean place to prevent bacterial growth on the sexual aids and subsequent vaginal or perineal infection. Therefore, it is impossible for women to completely rest and relax after masturbating with a sexual aid. All the problems mentioned above have urged the sexual aid industry to provide an effective solution or, more particularly, to design and produce a female sexual aid which can be carried conveniently without being noticed and which requires no cleanup after use.

### BRIEF SUMMARY OF THE INVENTION

In view of the shortcomings of the conventional female sexual aids, namely difficulty of carriage and inconvenient cleanup after use, the inventor of the present invention conducted extensive research, performed related experiments, and finally succeeded in developing a self-inflating sexual aid as disclosed herein, whose overall structure is designed to relieve women of their mental burden while carrying and after using a sexual aid, thereby effectively overcoming the drawbacks of the existing female sexual aids.

It is an objective of the present invention to provide a self-inflating sexual aid which can be folded and downsized when not in use so as to facilitate carriage and storage and which can be directly discarded after use so as to spare its user the otherwise necessary cleanup work. The sexual aid includes a closed bag which is formed by hot-pressing and sealing at least one film such that the closed bag has an edge formed as an airtight sealed edge while a receiving space is formed between the at least one film. Inside the receiving space are a reactive powder (e.g., a compound containing carbonate ions, such as sodium carbonate, sodium bicarbonate, and baking powder) and a sachet containing a reactive liquid (e.g., edible citric acid, acetic acid, and diluted hydrochloric acid). When squeezed, the sachet is easily broken at an edge thereof, such that the reactive liquid flows out of the sachet into the receiving space. A chemical reaction takes place when the reactive liquid contacts with the reactive powder. As a result, gaseous carbon dioxide is produced, and the closed bag is automatically and instantly inflated to a completely taut state, in which the closed bag has an end inflated into a rod-shaped portion and an opposite end inflated into a handle portion. In addition, an elastic cover has an end formed with an opening and an opposite end that is sealed. The elastic cover has an inner surface that can be fitted closely over an outer surface of the rod-shaped portion of the closed bag, thus forcing a portion of the airtight sealed edge that corresponds to the rod-shaped portion to lie smoothly against the outer surface of the rod-shaped portion. Consequently, a user can hold the handle portion of the closed bag and use the sexual aid by maneuvering the rod-shaped portion of the closed bag.

It is another objective of the present invention to provide the foregoing sexual aid, wherein the closed bag, when not inflated, can be stored in a cosmetic bag without taking any extra storage space. Furthermore, the uninflated closed bag does not have a three-dimensional phallic shape and therefore is unlikely to be identified; in other words, the sexual aid is highly inconspicuous. When it is desired to use the sexual aid, the user only has to squeeze and thereby break the sachet, allowing the outflowing reactive liquid to react with the reactive powder, such that carbon dioxide is produced to inflate the closed bag into a three-dimensional phallic shape automatically and instantly. After the elastic cover is fitted over the rod-shaped portion, the sexual aid can be safely used, without the risk that the user might be cut by the airtight sealed edge of the closed bag; in other words, the sexual aid provides high safety. After the sexual aid is used, the closed bag can be directly torn open to release the gas inside. Then, the sexual aid can be discarded without further cleaning. Hence, the sexual aid is effectively enhanced in convenience and hygiene.

### BRIEF DESCRIPTION OF THE SEVERAL VIEWS OF THE DRAWINGS

The invention as well as a preferred mode of use, further objectives, and advantages thereof will be best understood by referring to the following detailed description of an illustrative embodiment in conjunction with the accompanying drawings, in which:

FIG. 1 is a schematic view of a closed bag according to the present invention, wherein the closed bag is yet to be inflated;

FIG. 2 is a perspective view of the closed bag and an elastic cover according to the present invention, wherein the closed bag is inflated;

FIG. 3 is a schematic view of the closed bag according to the present invention, wherein the closed bag is folded; and

FIG. 4 is a sectional view of the closed bag and the elastic cover according to the present invention.

### DETAILED DESCRIPTION OF THE INVENTION

The present invention provides a self-inflating sexual aid which includes a closed bag, a reactive powder, a sachet containing a reactive liquid, and an elastic cover. The reactive powder and the sachet containing the reactive liquid are received hermetically in the closed bag. When the reactive powder contacts with the reactive liquid in the sachet, a gas is produced to inflate the closed bag into a three-dimensional phallic shape, and after the elastic cover is fitted over an outer surface of the closed bag, the sexual aid is ready for use. The closed bag can be torn open after use, thereby releasing the gas inside and allowing the sexual aid to be discarded. Compared with the prior art, the overall structure of the present invention provides more convenience and fun to women.

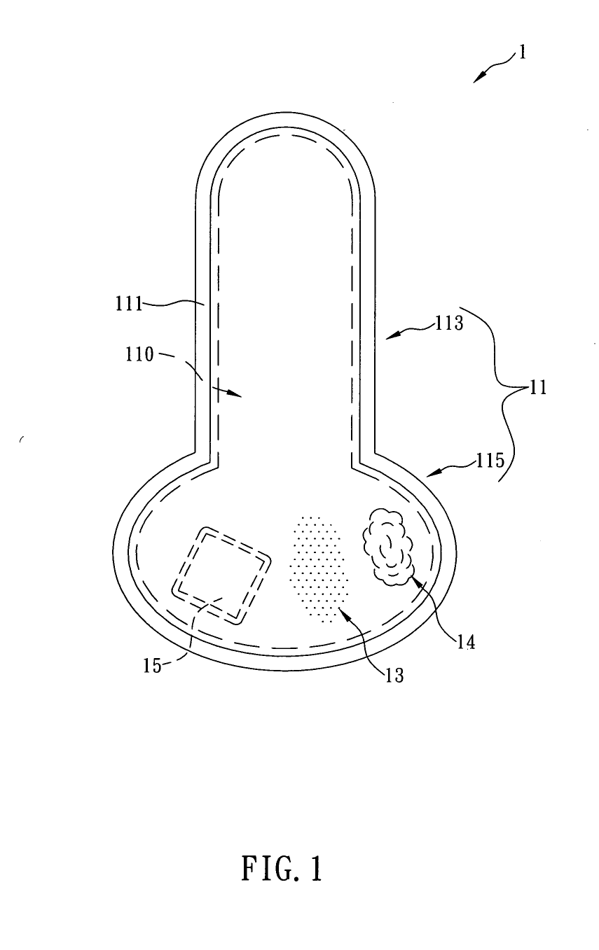

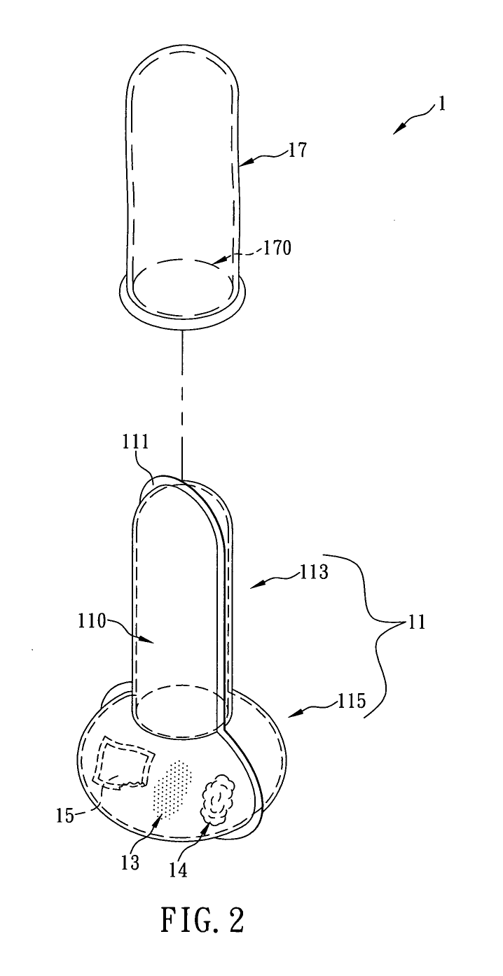

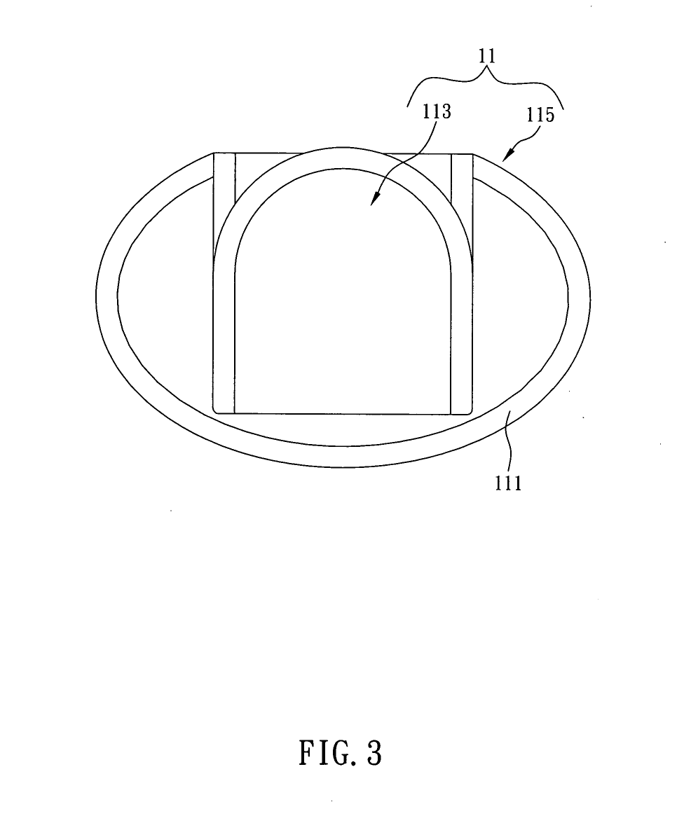

The technical features of the present invention are further described hereinafter by way of a preferred embodiment. Referring to FIG. 1, the closed bag 11 is made by hot-pressing and sealing at least one film. For instance, a manufacturer may fold a single film and then perform a hot-pressing and sealing process. Alternatively, the manufacturer may place one film on top of another and then carry out the hot-pressing and sealing process, thereby forming the closed bag 11. After the edge of the closed bag 11 is hot-pressed and sealed, an airtight sealed edge 111 is formed, and a receiving space 110 is formed between the at least one film. The closed bag 11 has an end formed as a rod-shaped portion 113 and an opposite end formed as a handle portion 115. It should be noted that the shape of the handle portion 115 as depicted in FIG. 1 is only exemplary. In another embodiment, the handle portion 115 may have a rectangular shape, a triangular shape, a circular shape, or a rod shape, provided that the handle portion 115 is shaped to facilitate gripping. The reactive powder 13 is a compound containing carbonate ions, such as sodium carbonate, sodium bicarbonate, and baking powder. The reactive powder 13 is hermetically sealed in the receiving space 110 and therefore will not be spread out of the closed bag 11 when the closed bag 11 is shaken. The sachet 15 containing the reactive liquid is also hermetically sealed in the receiving space 110. When a user squeezes the sachet 15 containing the reactive liquid through the closed bag 11, the sachet 15 is easily broken at the edge, as shown in FIG. 2, such that the reactive liquid (e.g., edible citric acid, acetic acid, or diluted hydrochloric acid) flows out of the sachet 15 into the receiving space 110 and contacts with the reactive powder 13. As a result, a chemical reaction takes place, and a gas is produced. For instance, acetic acid reacts with sodium bicarbonate to produce sodium acetate, water, and gaseous carbon dioxide. In this instance, as the gaseous carbon dioxide accumulates in the receiving space 110, the closed bag 11 is automatically and instantaneously inflated to a completely taut state, as shown in FIG. 2, thereby forming a three-dimensional shape that resembles a penis. Hence, when the closed bag 11 is not inflated, it can be folded and downsized as shown in FIG. 3 and easily put into a cosmetic bag for carriage. Since the folded closed bag 11 does not have a phallic shape, it is impossible for someone spotting the closed bag 11 to associate the object in sight with a sexual aid. Therefore, a female can carry the sexual aid 1 of the present invention in her cosmetic bag and be spared the embarrassment of others accidentally seeing and identifying the sexual aid 1 when she fumbles in the cosmetic bag. In other words, the sexual aid 1 of the present invention is effectively enhanced in terms of secrecy.

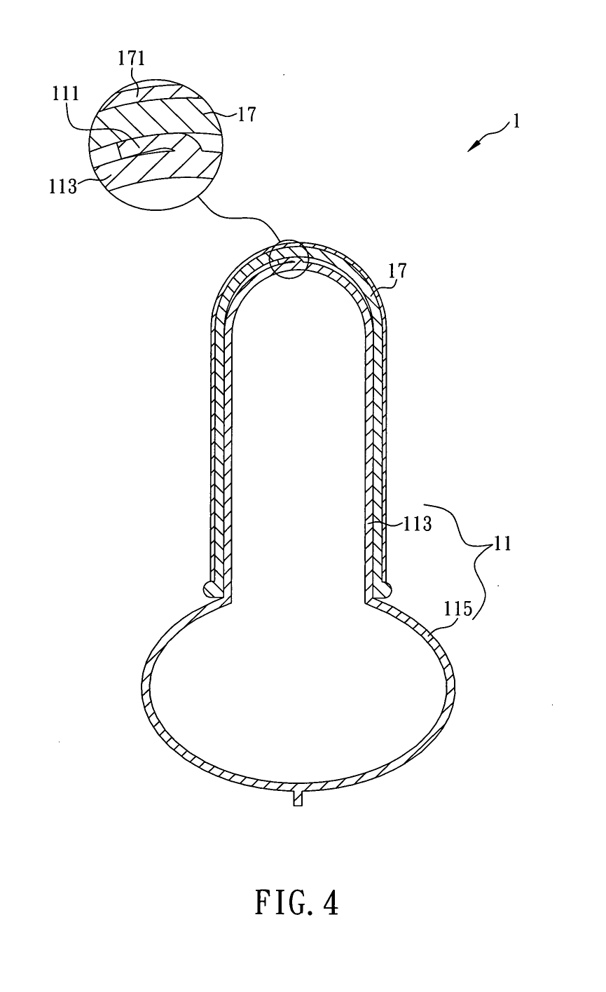

Referring to FIG. 2 and FIG. 4, the elastic cover 17 has an end formed with an opening 170 and an opposite end that is sealed, such that the elastic cover 17 can be completely fitted over an outer surface of the rod-shaped portion 113 of the closed bag 11 through the opening 170. The elastic cover 17 is provided mainly because the airtight sealed edge 111 of the closed bag 11 will stand upright along an outer periphery of the closed bag 11 after the closed bag 11 is inflated, thus posing a risk of injuring the user during use. However, once the elastic cover 17 is fitted over the outer surface of the rod-shaped portion 113 of the closed bag 11, the elastic cover 17 forces a portion of the airtight sealed edge 111 that corresponds to the rod-shaped portion 113 to lie flat against the outer surface of the rod-shaped portion 113. Then, the user can grip the handle portion 115 of the closed bag 11 and use the sexual aid 1 by maneuvering the rod-shaped portion 113 of the closed bag 11, while the airtight sealed edge 111 of the closed bag 11 is prevented from causing injury or any discomfort to the user. Furthermore, in order to increase convenience of use, a lubricant 171 is coated on an outer surface of the elastic cover 17 to provide the elastic cover 17 with an enhanced lubricating effect and thereby protect the user from abrasion attributable to dryness. Therefore, a female user can fold the uninflated closed bag 11 and carry it with her by storing it in a cosmetic bag, as shown in FIG. 3, in which case the closed bag 11 not only takes no extra storage space at all, but also is unidentifiable by others. When it is desired to use the sexual aid 1, the sachet 15 containing the reactive liquid is squeezed until broken, such that the reactive liquid flows out and reacts with the reactive powder 13, and the gaseous carbon dioxide thus produced automatically and instantaneously inflates the closed bag 11 into a three-dimensional phallic shape. Following that, the elastic cover 17 is fitted over the rod-shaped portion 113, and the sexual aid 1 is ready and safe to be used. After use, the closed bag 11 can be directly torn open, thereby releasing the gas inside, and be discarded without further cleaning.

Referring back to FIG. 1, the closed bag 11 further contains an absorbent material 14, such as cotton. When the sachet 15 containing the reactive liquid is squeezed and broken, the outflowing reactive liquid is absorbed by the absorbent material 14. Thus, when the closed bag 11 is torn open after use, the reactive liquid will not splash around and burn the user. Moreover, as the reaction between the reactive liquid and the reactive powder may produce water in addition to gaseous carbon dioxide, the absorbent material 14 also serves to absorb the water produced, so that the user's clothes or the user herself will not get wet from the liquid when tearing the closed bag 11 open, thereby saving the user from subsequent cleaning.

As stated previously, in today's society where medical science is flourishing, it has been acknowledged that autosexual behavior makes positive contributions to the physical and mental health of, for example, celibate, widowed, and handicapped women. The sexual aid 1 of the present invention can inflate itself automatically and instantly and is therefore highly erotic and entertaining, increasing the fun and quality of masturbation as well as marital sex life. Besides, the sexual aid 1 can be conveniently carried and directly discarded after use, thereby overcoming the shortcomings of the prior art, namely inconvenience in carriage and potential infection caused by incomplete cleaning and bacterial growth. In short, the sexual aid 1 of the present invention is highly inconspicuous, safe, and hygienic.

What is claimed is:

1. A self-inflating sexual aid, comprising:
a closed bag formed by hot-pressing and sealing at least a film such that an edge of the closed bag is hot-pressed and sealed and becomes an airtight sealed edge while a receiving space is formed between the at least a film, wherein the closed bag has an end formed as a rod-shaped portion and an opposite end formed as a handle portion;
a sachet containing a reactive liquid and hermetically sealed in the receiving space, wherein the sachet, upon being squeezed, is easily broken at an edge thereof, thus allowing the reactive liquid to flow out of the sachet into the receiving space;
a reactive powder hermetically sealed in the receiving space, wherein when the reactive powder contacts with the reactive liquid, gaseous carbon dioxide is produced to automatically and instantly inflate the closed bag into a three-dimensional phallic shape; and
an elastic cover having an end formed with an opening and an opposite end sealed, wherein the elastic cover has an inner surface to be fitted tightly over an outer surface of the rod-shaped portion of the closed bag such that a portion of the airtight sealed edge of the closed bag that corresponds to the rod-shaped portion lies flat against the outer surface of the rod-shaped portion.

2. The sexual aid of claim 1, wherein the elastic cover has an outer surface coated with a lubricant.

3. The sexual aid of claim 1, further comprising an absorbent material hermetically sealed in the receiving space and capable of absorbing the reactive liquid in the sachet.

4. The sexual aid of claim 2, further comprising an absorbent material hermetically sealed in the receiving space and capable of absorbing the reactive liquid in the sachet.
